(19) United States
(12) Patent Application Publication
(10) Pub. No.: US 2020/0222601 A1
(43) Pub. Date: Jul. 16, 2020

(54) BLOOD SEPARATION METHOD

(71) Applicant: AEON BIOTHERAPEUTICS CORP., Taipei City (TW)

(72) Inventors: DAI-JEN LEE, Taipei City (TW); SHIH-WEI CHEN, Taipei City (TW)

(21) Appl. No.: 16/737,306

(22) Filed: Jan. 8, 2020

(30) Foreign Application Priority Data
Jan. 11, 2019 (TW) 108101218

(51) Int. Cl. A61M 1/02; A61M 1/36; A61K 35/19; A61K 35/16

(52) U.S. Cl. CPC A61M 1/0272; A61M 1/029; A61K 35/16; A61K 35/19; A61M 1/3695

(57) ABSTRACT

A blood separation method is provided. The blood separation method includes the following steps. Firstly, a whole blood is placed in a container having a separating gel, which includes a silicon-containing polymer, and a specific gravity of the separating gel is between 1.030 and 1.093. Then, a centrifugation is performed at a first effective rotation speed to divide the whole blood into a red blood cell layer located below the separating gel, a buffy coat layer located above the separating gel, and a plasma layer located above the buffy coat layer. The buffy coat layer and the plasma layer are then mixed to obtain a platelet-rich plasma.

### CROSS-REFERENCE TO RELATED PATENT APPLICATION

This application claims the benefit of priority to Taiwan Patent Application No. 108101218, filed on Jan. 11, 2019. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a blood separation method, and more particularly to a method for quickly separating blood.

### BACKGROUND OF THE DISCLOSURE

With the advancement of regenerative medicine, the technology of using stem cells and growth factors to promote the repair and regeneration of cellular tissues to achieve therapeutic effects has matured significantly.

Platelet-rich plasma (PRP), also known as high-concentration platelet plasma, is obtained by centrifuging whole blood. The main active component of PRP is platelets. Many granules, mainly growth factors, with different functions will be released when platelets are activated, which can stimulate cell proliferation and differentiation, and have very good results in alleviating tissue inflammation and promoting cell tissue repair and proliferation. Platelet-rich plasma has been used clinically in the sports medicine, such as to treat soft tissue injuries of athletes, and can also be used in the aesthetic medicine and dentistry. Moreover, platelet-containing plasma is usually prepared by collecting autologous blood, so that it does not cause rejection of non-autologous tissue.

In addition, platelet-rich fibrin (PRF), which is called second-generation PRP, is jelly-like and has good plasticity. PRF is often used in conjunction with bone graft in alveolar bone regeneration surgery, or is applied to periodontal regeneration surgery after being compressed into a membrane form.

Compared with the above-mentioned applications, platelet membranes, fibrin, cell debris, etc., are removed from platelet lysate (PL), which can reduce an immune response while retaining growth factors. Studies have pointed out that platelet lysate can significantly promote bone regeneration and repair, and the mixture of platelet lysate and autologous bone marrow mesenchymal stem cells can accelerate bone formation.

However, since platelet-rich plasma, platelet-rich fibrin, and platelet lysate are usually applied immediately after separation, studies have pointed out that residual red blood cells may cause pain and inflammatory responses in the recipient. Therefore, how to quickly and efficiently separate platelet-rich plasma, platelet-rich fibrin, or platelet lysate with high-concentration active ingredients is an important issue yet to be solved in this field.

### SUMMARY OF THE DISCLOSURE

In response to the above-referenced technical inadequacies, the present disclosure provides a blood separation method, which can quickly prepare a platelet-rich plasma or platelet lysate in the same separation apparatus and has a high red blood cell removal rate.

In one aspect, the present disclosure provides a blood separation method including the following steps. Firstly, a whole blood is placed in a container having a separating gel, which includes a silicon-containing polymer. A specific gravity of the separating gel is between 1.030 and 1.093. Then, a centrifugation is performed at a first effective rotation speed to divide the whole blood into a red blood cell layer located below the separating gel, a buffy coat layer located above the separating gel, and a plasma layer located above the buffy coat layer. The buffy coat layer and the plasma layer are then mixed to obtain a platelet-rich plasma.

In another aspect, the present disclosure provides a blood separation method including the following steps. Firstly, a whole blood is placed in a container having a separating gel, which includes a silicon-containing polymer, and a specific gravity of the separating gel is between 1.030 and 1.093. Then, a centrifugation is performed at a first effective rotation speed to divide the whole blood into a red blood cell layer located below the separating gel, a buffy coat layer located above the separating gel, and a plasma layer located above the buffy coat layer. The buffy coat layer and the plasma layer are then mixed to obtain a platelet-rich plasma. Lastly, a multi-stage centrifugation is performed at a second effective rotation speed so that the platelet-rich plasma forms a platelet lysate.

One of the advantages of the present disclosure is that, the blood separation method of the present disclosure can quickly separate the platelet-rich plasma or platelet lysate in the same container through the technical solution of “a centrifugation at the first effective rotation speed to divide the whole blood into the red blood cell layer, a buffy coat layer, and a plasma layer by the separating gel” and “the separating gel includes a silicon-containing polymer, and a specific gravity of the separating gel is between 1.030 and 1.093”.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 is a flowchart of a blood separation method of the present disclosure.

FIG. 2 is a side view of a separation apparatus used in the blood separation method of the present disclosure.

FIG. 3 is a schematic view of step S100 in FIG. 1.

FIG. 4 is a schematic view of step S102 being completed in FIG. 1.

FIG. 5 is a schematic view of step S104 being completed in FIG. 1.

FIG. 6 is a schematic view of step S106 in FIG. 1.

FIG. 7 is a schematic view of step S108 in FIG. 1 being completed.

### DETAILED DESCRIPTION OF THE EXEMPLARY EMBODIMENTS

In recent years, due to the increasing demand for autologous growth factor regenerative therapies, there is an urgent need for a way of effectively and quickly separating high concentrations of platelet-rich plasma (PRP), platelet-rich fibrin (PRF), and platelet lysate (PL). In addition, it is necessary to ensure that patients would not feel pain or have inflammatory reactions due to residual red blood cells in PRP, PRF, or PL during an autologous regenerative treatment. Therefore, the present disclosure provides a blood separation method, which can directly prepare a platelet-rich plasma or platelet lysate in the same separation apparatus and has a high red blood cell removal rate.

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

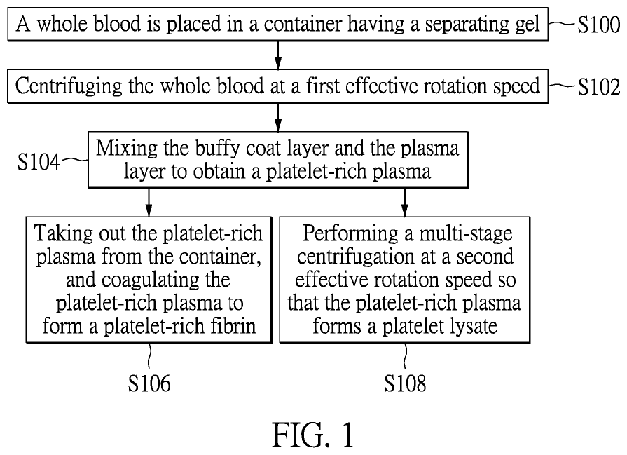

Referring to FIG. 1, an embodiment of the present disclosure provides a blood separation method, which includes the following steps: Step S100, placing a whole blood in a container having a separating gel; Step S102, centrifuging the whole blood at a first effective rotation speed to divide the whole blood into a red blood cell layer, a buffy coat layer, and a plasma layer; and Step S104, mixing the buffy coat layer and the plasma layer to obtain a platelet-rich plasma.

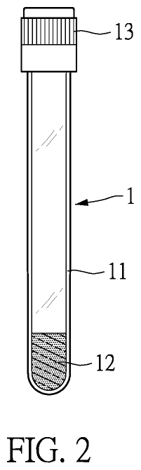

Referring to FIG. 2, a separation apparatus 1 used in the present disclosure includes a container 11, a separating gel 12, and a cap 13. The container 11 is a rigid tube with an open end and a closed end, and the separating gel 12 is placed in the container 11. The separating gel 12 can, for instance, be placed near the closed end. The cap 13 is detachably and tightly engaged with the open end of the container 11 to expose or cover the open end. It should be noted that, the separating gel 12 includes a silicon-containing polymer, and the specific gravity of the separating gel 12 is between 1.030 and 1.093, but is not limited thereto. In this embodiment, the separating gel 12 can further include other components as needed, e.g., a thixotropic agent and a non-toxic inert organic lubricant. Specifically speaking, the thixotropic agent is used to adjust the viscosity of the separating gel 12. The physical properties of the separating gel 12 are shown in Table 1 below.

TABLE 1
The physical properties of the separating gel 12

| | |
|---|---|
| Specific gravity | 1.030-1.093 |
| Viscosity | <40 × 10⁴ mPa · s |
| Bubble test | No bubbles generated after a |
| | centrifugation |
| Centrifugal test | Colloidal flip thickness >5 mm |
| Low specific gravity test | gel does not flip |
| Volatility | <1.0% |

For instance, the separation apparatus 1 can be a plastic centrifuge tube or a glass centrifuge tube, but is not limited thereto. In operation, after blood collection, the whole blood is transferred into the separation apparatus 1 in an aseptic condition, and the separation apparatus 1 can be placed in a centrifuge and be centrifuged.

Again, referring to FIG. 1, the embodiment of the present disclosure provides the blood separation method, which can further include: Step S106, taking out the platelet-rich plasma from the container, and coagulating the platelet-rich plasma to form a platelet-rich fibrin.

Alternatively, the embodiment of the present disclosure provides the blood separation method, which can further include: Step S108, performing a multi-stage centrifugation at a second effective rotation speed so that the platelet-rich plasma forms a platelet lysate.

It is worth mentioning that, the platelet-rich plasma obtained in Step S104, the platelet-rich fibrin obtained in Step S106, and the platelet lysate obtained in Step S108 are all target products belonging to the blood separation method of the present disclosure. The method of separating each target product will be described in detail below.

FIG. 1 is a flowchart of the steps of the blood separation method of the present disclosure, and FIG. 1 can be referred to for the following preparation examples.

### First Preparation Example

### Preparation of Platelet-Rich Plasma (PRP)

In the blood separation method of the present disclosure, the whole blood can be collected from an autologous blood of mammalian. Therefore, after obtaining autologous regeneration-promoting substances (e.g., platelet-rich plasma, platelet-rich fibrin, and platelet lysate), it can be applied to autologous treatment afterwards without causing rejection from an immune response.

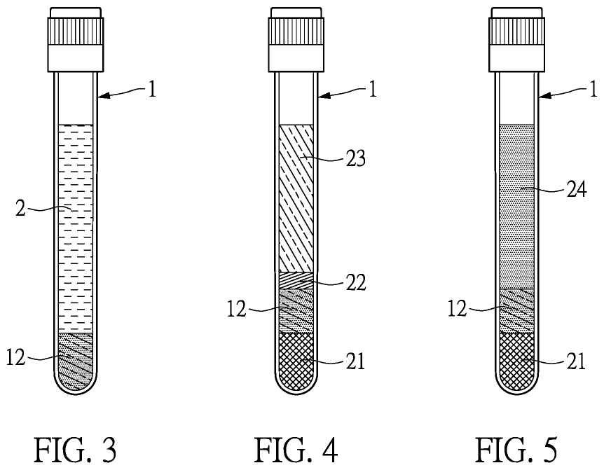

Referring to Step S100 of FIG. 1, the whole blood is placed in the container having a separating gel. Referring to FIG. 3, in this preparation example, 10 ml of a whole blood 2 of a recipient is firstly placed into the separation apparatus 1. Generally, the separating gel 12 can be filled in the bottom of the container 11 with a thickness greater than 5 mm, but is not limited thereto. The vacuum blood collection tube may be used for collecting the whole blood, or the blood in the vacuum blood collection tube may be aseptically placed in the separation apparatus 1.

Referring to Step S102 of FIG. 1, centrifuging the whole blood at a first effective rotation speed. In this preparation example, the separation apparatus 1 containing the whole blood 2 is centrifuged at the first effective rotation speed. The first effective rotation speed can be, but is not limited to being, between 3000 rpm and 4000 rpm, and the centrifugation time may be, but is not limited to being, between 3 minutes and 10 minutes. After a centrifugation, as shown in FIG. 5, the separation apparatus 1 will have a red blood cells layer 21, a separating gel 12, a buffy coat layer 22, and a plasma layer 23 from bottom to top, with a total of four separated layers.

The main content of the red blood cell layer 21 is red blood cells, which accounts for about 40% of the total volume of the whole blood 2 and is responsible for the transportation of oxygen and carbon dioxide. In the whole blood 2, only red blood cells have a larger specific gravity than that of the separating gel 12. Therefore, during a centrifugation, only red blood cells can pass through the separating gel 12 and settle in the lowest layer of the separation apparatus 1, so that the separating gel 12 is located between the red blood cell layer 21 and the buffy coat layer 22, that is, the separating gel 12 isolates the red blood cell layer 21.

The main contents of the buffy coat layer 22 are nucleated cells (e.g., stem cells and white blood cells) and platelets, which account for about 5% of the total volume of the whole blood 2. The main content of the plasma layer 23 is the serum component of the whole blood 2, which accounts for about 55% of the total volume of the whole blood 2. The plasma layer 23 contains water and many proteins, e.g., antibodies, enzymes, hormones, and so on.

Lastly, referring to Step S104 of FIG. 1, the buffy coat layer and the plasma layer are mixed to obtain a platelet-rich plasma. The buffy coat layer 22 and the plasma layer 23 are well mixed in the separation apparatus 1 so as to obtain a platelet-rich plasma 24 (as shown in FIG. 5).

Furthermore, before mixing the buffy coat layer 22 and the plasma layer 23, 1 to 90% by volume of the total volume of the plasma layer 23 can be removed, and the remaining plasma layer 23 can be retained, so that a high concentration from 1.3 to 25 times of platelet-rich plasma can be obtained at Step S104. For instance, 4 ml of the plasma layer 23 that was originally 5 ml after a centrifugation is taken, with about 1 ml of the plasma layer 23 being retained, and then the buffy coat layer 22 and the plasma layer 23 is mixed to obtain a concentrated platelet-rich plasma 24, but the present disclosure is not limited thereto. For instance, during a surgical operation, the doctor will evaluate and decide on the total amount of PRP to be applied according to the affected part, and then calculate the remaining volume of the plasma layer 23 that is required.

It is worth mentioning that, the separating gel 12 does not flip over as the separation apparatus 1 is tilted upside down. Therefore, the blood separation method of the present disclosure can directly perform the step of mixing the buffy coat layer 22 and the plasma layer 23 in the same separation apparatus 1, and then obtain the platelet-rich plasma 24. The mixing can be performing repeated suction by using an electric pipette aid or a micro-dispenser, or after the cap 13 of the separation apparatus 1 is tightly closed, the two ends of the separation apparatus 1 are repeatedly tilted for mixing. During the mixing, because the separating gel 12 isolates the red blood cell layer 21, the red blood cell layer 21 will not be mixed into the platelet-rich plasma 24 from repeated tilting.

According to research, when red blood cells remain in autologous regeneration-promoting substances (e.g., platelet-rich plasma, platelet fibrin, and platelet lysate), it may cause patients to feel pain or have inflammation and other reactions. However, a common blood separation method can only divide whole blood, and as a result, the red blood cell layer will be close to the buffy coat layer. Part of the buffy coat layer must be discarded to avoid the red blood cells, which leads to plasma having lower platelet content. In other words, in order to obtain platelet-rich plasma with high platelet content, the maximum amount possible of the plasma layer and buffy coat layer needs to be taken, but in this process, red blood cells may also be taken during the process, so that the risk of residual red blood cells and the risk of side effects will be also higher. Further, since the buffy coat layer is adjacent to the red blood cell layer, it is necessary to move the plasma layer and the buffy coat layer to another container so that the plasma layer and the buffy coat layer can be mixed, which complicates the operation steps and increases the working time. The blood separation method of the present disclosure requires only less than 10 minutes of operation to complete the preparation of autologous platelet-rich plasma.

It should be noted that, because anticoagulants will inhibit platelet activation, when platelets are in an inactive state, the production of growth factors secreted by platelets will be greatly reduced. The blood separation method of the present disclosure does not contain any anticoagulant. Therefore, compared to the blood separation method containing an anticoagulant, the growth factor concentrations of the platelet-rich plasma, platelet-rich fiber, and platelet-rich lysate prepared by the blood separation method of the present disclosure can be 2.6 to 13 times higher than the blood separation method containing an anticoagulant.

### Second Preparation Example

### Preparation of Platelet-Rich Fibrin (PRF)

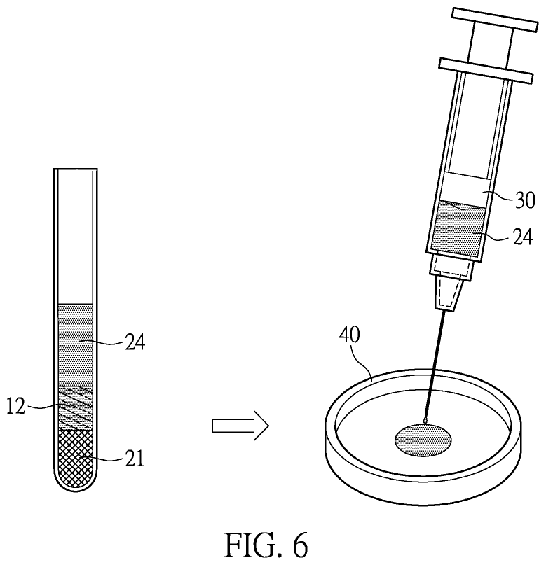

Referring to FIG. 1, the blood separation method of the present disclosure can further include Step S106, which is taking out the platelet-rich plasma from the container, and coagulating the platelet-rich plasma to form a platelet-rich fibrin. Referring to FIG. 6, the platelet-rich plasma 24 obtained from the first preparation example is taken out by using a syringe 30 and placed in a shaping dish 40 and let sit for a period of time. Said period of time is determined by the patient's physical condition and previous medications, and is generally for, but not limited to, 15 to 45 minutes, before the platelet-rich plasma 24 will naturally coagulate to from a platelet-rich fibrin. Further, a platelet-rich fibrin film can also be obtained by extruding platelet-rich fibrin with a sterile gauze.

Furthermore, platelet-rich fibrin can also be prepared from the concentrated platelet-rich plasma 24.

It is worth mentioning that, the shaping dish 40 can be adjusted according to the requirements of the operation or application, so that platelet-rich fibrin can be shaped along with the shape of the shaping dish 40. Therefore, the platelet-rich fibrin is very widely applicable in medical surgeries.

### Third Preparation Example

### Preparation of Platelet Lysate (PL)

Platelet lysate is the liquid portion of platelets after lysis, which retains many growth factors and removes platelet membranes and other cell debris, which can further reduce the immune response generated during transplantation and regeneration.

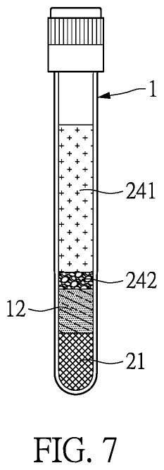

Referring to FIG. 1, the blood separation method of the present disclosure can further include Step S108, which is performing a multi-stage centrifugation at a second effective rotation speed to make the platelet-rich plasma form a platelet lysate. The platelet-rich plasma 24 obtained from the first preparation example is retained in the separation apparatus 1 and a multi-stage centrifugation is performed to divide the platelet-rich plasma 24 into an upper separation liquid 241 and a lower layer separation liquid 242 (as shown in FIG. 7). The upper separation liquid 241 is platelet lysate, and the lower separation liquid 242 is composed of platelet fibrin, platelet membrane, and other cell debris. Specifically speaking, the second effective rotation speed of the multi-stage can be, but is not limited to being, between 3500 rpm and 4500 rpm, and the centrifugation time may be, but is not limited to being, between 3 minutes and 15 minutes. In addition, the multi-stage centrifugation includes performing an intermittent centrifugation at a predetermined interval, for instance, the intermittent centrifugation is repeated 5 to 15 times according to the second effective rotation speed, and the number of times of repeated centrifugation is based on whether the platelet-rich plasma 24 divides the upper and lower layer separation fluid.

### Applications of PRP, PRF and PL

The platelet-rich plasma, the platelet-rich fibrin, and the platelet lysate prepared by the above-mentioned blood separation methods are rich in many growth factors and can be used in regenerative treatments of dental, orthopedics, rehabilitation, aesthetic medicine, and obstetrics and gynecology and so on. Further, the growth factors include insulin-like growth factor (IGF), keratinocyte growth factor (KGF), epidermal growth factor (EGF), transforming growth factor (TGF), fibroblast growth factor (FGF), vascular endothelial growth factor (VEGF), platelet derived growth factor (PDGF), and connective tissue growth factor (CTGF), and so on, but not limited to thereto. The main functions of the growth factor include stimulating cell proliferation, stimulating cell migration, helping wound healing, promoting angiogenesis, and promoting collagen secretion.

For instance, the blood separation method of the present disclosure can be performed to obtain the autologous platelet-rich plasma. The autologous platelet-rich plasma can then be mixed with hyaluronic acid, and injected into the patient's dermis and subcutaneous layer so as to achieve the effect of face modification and to eliminate static wrinkles.

For another instance, for a patient with alveolar bone atrophy, an autologous platelet-rich fibrin can be obtained through the blood separation method of the present disclosure. Then, the platelet-rich fibrin is dipped in bone graft, and then filled into the alveolar bone of the patient to help with osteoblast proliferation and accelerate wound healing.

For another instance, for a patient suffering from arthritis, the blood separation method of the present disclosure can be used to obtain an autologous platelet lysate, and the arthritis can then be treated by injecting the affected part to help with healing of the arthritis.

### Beneficial Effects of the Embodiments

One of the advantages of the present disclosure is that, the blood separation method of the present disclosure can quickly separate platelet-rich plasma or platelet lysate in the same container through the technical solution of “a centrifugation at the first effective rotation speed to divide the whole blood into the red blood cell layer, a buffy coat layer, and a plasma layer by the separating gel” and “the separating gel includes a silicon-containing polymer, and a specific gravity of the separating gel is between 1.030 and 1.093”.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A blood separation method, comprising:
(a) placing a whole blood in a container having a separating gel, wherein the separating gel includes a silicon-containing polymer, and a specific gravity of the separating gel is between 1.030 and 1.093;
(b) centrifuging the whole blood at a first effective rotation speed to divide the whole blood into a red blood cell layer located below the separating gel, a buffy coat layer located above the separating gel, and a plasma layer located above the buffy coat layer; and
(c) mixing the buffy coat layer and the plasma layer to obtain a platelet-rich plasma.

2. The blood separation method according to claim 1, wherein after step (c), the method further comprises the step of taking out the platelet-rich plasma from the container, and coagulating the platelet-rich plasma to form a platelet-rich fibrin.

3. The blood separation method according to claim 1, wherein between steps (b) and (c), the method further comprises the step of removing 1% to 90% by volume of a total volume of the plasma layer so as to increase the concentration of the platelet-rich plasma by 1.3 to 25 times.

4. The blood separation method according to claim 2, wherein between steps (b) and (c), the method further comprises the step of removing 1% to 90% by volume of the total volume of the plasma layer so as to increase the concentration of the platelet-rich plasma by 1.3 to 25 times.

5. The blood separation method according to claim 1, wherein in step (b), the first effective rotation speed is from 3000 rpm to 4000 rpm, and the centrifugation time is from 3 to 10 minutes.

6. A blood separation method, comprising:
(a) placing a whole blood in a container having a separating gel, wherein the separating gel includes a silicon-containing polymer, and a specific gravity of the separating gel is between 1.030 and 1.093;
(b) centrifuging the whole blood at a first effective rotation speed to divide the whole blood into a red blood cell layer located below the separating gel, a buffy coat layer located above the separating gel, and a plasma layer located above the buffy coat layer;
(c) mixing the buffy coat layer and the plasma layer to obtain a platelet-rich plasma; and
(d) performing a multi-stage centrifugation at a second effective rotation speed so that the platelet-rich plasma forms a platelet lysate.

7. The blood separation method according to claim 6, wherein in step (b), the first effective rotation speed is from 3000 rpm to 4000 rpm, and the centrifugation time is from 3 to 10 minutes.

8. The blood separation method according to claim 6, wherein in step (d), the second effective rotation speed of the multi-stage centrifugation is from 3500 rpm to 4500 rpm, and the centrifugation time is from 3 to 15 minutes.

9. The blood separation method according to claim 6, wherein between steps (b) and (c), the method further comprises the step of removing 1% to 90% by volume of a total volume of the plasma layer so as to increase the concentration of the platelet lysate by 1.3 to 25 times.

10. The blood separation method according to claim 8, wherein the multi-stage centrifugation includes performing an intermittent centrifugation at a predetermined interval.

11. The blood separation method according to claim 10, wherein the intermittent centrifugation is repeated 5 to 15 times according to the second effective rotation speed.
